(19) United States
(12) Patent Application Publication
(10) Pub. No.: US 2024/0241113 A1
(43) Pub. Date: Jul. 18, 2024

(54) Method for detecting binding to an ADP-ribosyl group or a polymer thereof and a kit for performing said method

(71) Applicant: Oulun Yliopisto, Oulun Yliopisto (FI)

(72) Inventors: Lari Lehtiö, Oulu (FI); Sven Sowa, Oulu (FI); Albert Galera-Prat, Oulu (FI)

(21) Appl. No.: 18/289,576

(22) PCT Filed: May 6, 2022
(86) PCT No.: PCT/FI2022/050305

§ 371 Date: Nov. 6, 2023

(30) Foreign Application Priority Data
May 6, 2021 (FI) 20215539

(51) Int. Cl. G01N 33/542; C07K 14/47; C12Q 1/48

(52) U.S. Cl. CPC G01N 33/542; C07K 14/4702; C12Q 1/48; C07K 2319/60; C12Y 204/0203; G01N 2333/235; G01N 2333/91142; G01N 2440/38

(57) ABSTRACT

The present invention is directed to a method, kit, system and fusion protein for detecting binding to an ADP-ribosyl group or a polymer thereof, wherein said group or polymer is coupled to a peptide or protein, the method comprising the steps of: i) providing a first entity comprising a first label or tag, said entity comprising an amino acid sequence comprising a cysteine residue whereto at least one ADP-ribosyl group or an analog thereof is coupled via an S-glycosidic bond; ii) contacting in an assay said first entity with a second entity, said second entity being or suspected of being capable of binding to an ADP-ribosyl group or polymer thereof coupled to a peptide or protein; and iii) measuring a signal derived from said first label or localized by said tag, wherein the signal detected is different or is localized differently when said second entity binds to said at least one ADP-ribosyl group of the first entity from the signal detected when the binding interaction between said second entity and said ADP-ribosyl group has not occurred. The kit of the present invention provides means to perform the method of the invention.

### FIELD OF THE INVENTION

The present invention relates to assay technologies for the detection of interactions between biomolecules. Particularly, the present invention relates to the identification of proteins interacting with the ADP-ribosylation in cancer-related pathways and viral infections. An aim of the present invention is to provide a robust assay technology suitable for high-throughput screening for a wide range of hydrolysing and non-hydrolysing ADP-ribose binders.

### BACKGROUND OF THE INVENTION

ADP-ribosylation is a post-translational modification involved in the regulation of many diverse processes in the cell. Despite its physiological importance, the intricate interplay of ADP-ribose transfer, detection and removal are not well understood on the molecular level.

The human genome encodes many different binders (“readers”) of ADP-ribosyl modified proteins as well as proteins that are able to hydrolyse and remove the ADP-ribosyl groups (“erasers”) (Teloni & Altmayer, 2016). Macrodomains represent one of the largest class of ADP-ribose binders known in humans. Many of them are encoded as part of other multidomain proteins such ADP-ribosyl-transferases (PARP9, PARP14, PARP15) or histones (macroH2A variants). Other macrodomains such as MDO1, MDO2, PARG or TARG1 possess hydrolysis activity and are integral actors in ADP-ribose signalling pathways.

Several other domain types exist that are primarily associated with binding of poly-ADP-ribose. Examples of these are the PAR-binding zinc finger (PBZ) domain of APLF, WWE domain in multiple ADP-ribosyl-transferases and E3 ubiquitin ligases and PAR binding motif (PBM) of XRCC1 (Teloni & Altmayer, 2016).

Macrodomains of pathogenic viruses represent another class of ADP-ribose binders. Viruses such as coronaviruses or togaviruses are known to harbour macrodomains that can remove ADP-ribose from proteins inside the host cell. These viral macrodomains are implied to weaken the host virus defence mechanism by interfering with the host ADP-ribosylation signalling machinery and have been shown to be necessary for virus replication and pathogenesis. Viruses with these macrodomains include Chikungunya virus, MERS-CoV (camel flu) and SARS-CoV-2 (COVID-19).

While development of inhibitors for ADP-ribosyl-transferases has been investigated since multiple decades mainly in the context of cancer therapeutics, the development of inhibitors against binders or hydrolysers of ADP-ribose has only gained momentum in recent years. Inhibitors of ADP-ribose binding proteins would be valuable tools that could help to decipher the complex ADP-ribosyl signalling machinery inside the cell. These inhibitors might also display therapeutic potential. Furthermore, the inhibition of viral macrodomains to combat diseases caused by these viruses is currently being explored.

Previous assays determining the hydrolysis of ADP-ribosylated substrate proteins cannot be directly applied to non-hydrolysing binders of ADP-ribose (Wazir et al., 2021). As the known assays for ADP-ribose binding are complex, expensive or not suited for high-throughput processes, there is still a need in the field for improvements.

### SUMMARY OF THE INVENTION

The aim of the present invention was to design a system that enables easy development of an assay suited for detection of a wide variety of ADP-ribosyl-binders and -hydrolases alike. The inventors reasoned that a non-hydrolysable ADP-ribose probe could be used to measure the binding of both hydrolysing- and non-hydrolysing ADP-ribose binders.

In proteins, many different residues can serve as acceptors of ADP-ribose. Residues such as serine, aspartate or glutamate form an O-glycosidic bond and lysine, arginine or asparagine form an N-glycosidic bond with ADP-ribose. Additionally, a less common modification is the chemically stable linkage via an S-glycosidic bond that can be formed with cysteine residues. While many different ADP-ribosyl-hydrolases exist and can remove ADP-ribose from O- or N-glycosidic bonds, to date there is no enzyme in humans reported able to reverse the S-glycosidic linkage.

Pertussis toxin from the bacterium Bordetella pertussis is known to efficiently catalyse the transfer of an ADP-ribose unit to a specific C-terminal cysteine residue in the αi subunits of heterotrimeric G proteins (Gαi). As an example to show how the invention works, we reduced Gαi to an 8-mer peptide and recombinantly fused it to the C-terminus of other proteins and still observed efficient modification, allowing site-specific addition of an ADP-ribose unit to any protein with accessible C-terminus. We used this to generate a MARylated YFP protein with a stable S-glycosidic bond able to bind ADP-ribose readers or erasers. We further extended this system by finding conditions to extend the MAR to PAR, allowing us to probe the binding of MAR and PAR binders alike.

Accordingly, the present invention provides a method for detecting binding to an ADP-ribosyl group or a polymer thereof, wherein said group or polymer is coupled to a peptide or protein, the method comprising the steps of:
i) providing a first entity comprising a first label or tag, said entity comprising an amino acid sequence comprising a cysteine residue whereto at least one ADP-ribosyl group or an analog thereof is coupled via an S-glycosidic bond;
ii) contacting in an assay said first entity with a second entity, said second entity being or suspected of being capable of binding to an ADP-ribosyl group or polymer thereof coupled to a peptide or protein; and
iii) measuring a signal derived from said first label or localized by said tag, wherein the signal detected is different or is localized differently when said second entity binds to said at least one ADP-ribosyl group of the first entity from the signal detected when the binding interaction between said second entity and said ADP-ribosyl group has not occurred.

In other related aspect, the present invention provides a kit for detecting binding to an ADP-ribosyl group or a polymer thereof, wherein said group or polymer is coupled to a peptide or protein, the kit comprising
in a first container: a first entity comprising a first label or tag, said entity comprising an amino acid sequence comprising a cysteine residue whereto at least one ADP-ribosyl group is coupled via an S-glycosidic bond, and
in a second container: a second entity coupled to a second label or tag, wherein said second entity is capable of binding to an ADP-ribosyl group or a polymer thereof coupled to a peptide or protein.

In other related aspect, the present invention provides a fusion protein comprising a first domain and a second domain, wherein said second domain comprises an amino acid sequence corresponding to the C-terminal sequence of a G alpha subunit of G proteins or having at least 75% sequence identity with the C-terminal sequence of a G alpha subunit of G proteins, preferably SEQ ID NO:4, and wherein said amino acid sequence comprises a cysteine residue whereto at least one ADP-ribosyl group or an analog thereof is coupled via an S-glycosidic bond.

In a further related aspect, the present invention provides a system comprising
i) a fusion protein comprising a first domain and a second domain, wherein said second domain comprises an amino acid sequence comprising a cysteine residue whereto at least one ADP-ribosyl group or an analog thereof is coupled via an S-glycosidic bond; and
ii) a biomacromolecule preferably selected from a group consisting of macro domains, ARH family proteins, BRCT domains, PAR binding zinc motifs, and WWE domains, wherein said biomacromolecule specifically recognizes said at least one ADP-ribosyl group or an analog thereof of said fusion protein;
wherein, in said system, said biomacromolecule is bound to said at least one ADP-ribosyl group or an analog thereof of said fusion protein so that said biomolecule and said fusion protein are linked together and form a coupled entity.

### BRIEF DESCRIPTION OF THE DRAWINGS

FIG. 1: A molecular toolbox for in vitro interaction studies and assay development of ADP-ribosyl binding proteins. (a) Site-specific ADP-ribosylation of a C-terminal Gαᵢ-based 10-mer peptide (GAP tag) by pertussis toxin subunit S1 (PtxS1) allows for generation of single S-glycosidically linked mono-ADP-ribosyl (MAR) groups. (b) The MAR group of the GAP-tag can be extended to a poly-ADP-ribosyl (PAR) group by PARP2. This system can be used to measure binding of proteins interacting with mono- or poly-ADP-ribosyl groups by FRET or other binding technologies. (c) The GAP-tag can be used for site-specific labelling with NAD⁺ analogs. (d) High-affinity ADP-ribosyl-binders fused to nanoluciferase (Nluc) can be used as luminescent probes for fast, sensitive and selective detection of mono- and poly-ADP-ribosylated proteins in blot-based methods.

FIG. 2: Initial development of toolkit components. (a) Testing ADP-ribosylation by PtxS1 with different Ga constructs. Unlabelled or YFP-fused full length Ga, constructs and GAP-tagged YFP were tested as cysteine-ADPr acceptors when treated with 50 nM (+) or 250 nM (++) PtxS1. As controls, buffer or YFP-GAP in which the acceptor cysteine was mutated to alanine were used. Reactions were blotted on a nitrocellulose membrane and detection was done using Nluc-eAf1521. (b) The MAR group in the GAP-tag can be extended to PAR by PARP2. 10 μM YFP-GAP or YFP-GAP(MAR) were mixed with 1 mM NAD⁺ and 400 nM (+) or 4 μM (++) PARP2 or buffer as control. The reactions were run on SDS-PAGE and visualized using coomassie blue or by western-blot and detection using Nluc-eAf1521 or Nluc-ALC1. (c) Detection of MAR and PAR by Nluc-eAf1521. (d) Selective detection of PAR by Nluc-ALC1. Dilution series of YFP-GAP(±MAR) or TNKS1(±PAR) were blotted on nitrocellulose membranes.

FIG. 3: The Gas-tag can be used to introduce site-specific modifications with NAD⁺ analogs. (a) Site-specific biotinylation of the GAP-tag. GAP-tagged YFP was mixed with NAD⁺ or 6-Biotin-17-NAD⁺ in absence or presence of PtxS1. The reactions were blotted on a nitrocellulose membrane and detection of biotin was done with Streptavidin-HRP. (b) YFP-GAP was MARylated by PtxS1 using NAD⁺ or 6-propargyladenine-NAD⁺ containing an alkyne group. The resulting proteins YFP-GAP(MAR) or YFP-GAP(MAR-alkyne) or buffer were mixed with Cy3-azide or Cy5-azide and the copper(I)-catalyzed alkyne-azide cycloaddition reaction was performed by addition of 5 mM sodium ascorbate, 300 μM CuSO₄ and 600 μM L-Histidine. The samples were incubated for 3 hours at room temperature and blotted on nitrocellulose membranes and color images were taken. Unreacted Cy3-azide or Cy5-azide was removed by washing of the membranes in TBS-T and color images as well as fluorescent images were taken.

FIG. 4: Testing interactions of reported and potential readers and erasers with YFP-GAP. (a) Interactions of CFP-fused potential and confirmed ADP-ribosyl binders with MARylated YFP-GAP. 1 μM CFP-fusion proteins were mixed with 5 μM YFP-GAP or with 5 μM YFP-GAP(MAR) in absence or presence of 200 μM ADP-ribose. The ratiometric FRET signals were measured. (b) Test of ADP-ribosyl removal from GAP tag. 10 μM YFP-GAP(MAR) were prepared in absence (−) or presence (+) of 1 μM CFP-fused proteins or 0.01 μM to 1 μM snake venom phosphodiesterase I (SVP). Samples were incubated for 24 hours at room temperature and blotted on a nitrocellulose membrane. Detection was done with Nluc-eAf1521. (c) Interactions of poly-ADP-ribosyl binders with PARylated YFP-GAP. 250 nM CFP-fusion proteins were mixed with 500 nM YFP or with 500 nM YFP-GAP(PAR) in absence or presence of 100 μM ADP-ribose or 2.5 μM automodified PARP2. The ratiometric FRET signals were measured. (d) Representative dose-response curve of 1 μM CFP-SARS-CoV nsp3 and 5 μM YFP-GAP(MAR) upon competition with ADP-ribose. (e) Representative dose-response curve of 250 nM CFP-ALC1 and 500 nM YFP-GAP(PAR) upon competition with PARylated PARP2.

FIG. 5: Various assay technologies can easily be utilized to detect ADPr binding. (a) Measurement of interaction by FRET. Ratiometric FRET signal of CFP-MDO2 and YFP-Gαi(MAR) in absence (control) or presence of 200 μM ADP-ribose as shown in FIG. 4a. (b) Measurement of interaction by BRET. Ratiometric BRET signal of Nluc-MDO2 and YFP-Gαi(MAR) in absence (control) or presence of 200 μM ADP-ribose. (c) Measurement of interaction by AlphaScreen. Biotinylated MDO2 and His-tagged MARylated Ga, were mixed with streptavidin donor beads and chelate acceptor beads in absence (control) or presence of ADP-ribose. The luminescence signal was detected upon excitation of donor beads. (d) Measurement of interaction by biolayer interferometry. His-tagged YFP-GAP(MAR) was bound to the optical sensor surface and the change of signal after association (0 sec) or dissociation (120 sec) of unlabelled MDO2 protein was determined in absence or presence of 3.16 μM ADP-ribose.

FIG. 6: Development of a screening assay based on the YFP-GAP(MAR) probe using CFP-tagged SARS-CoV-2 nsp3 macrodomain. (a) Signal validation for a screening assay with CFP-SARS-CoV-2. 1 μM SARS-CoV-2 was mixed with 5 μM YFP-GAP(MAR) in the absence or presence of 200 μM ADP-ribose and a Z′-factor of 0.7 was calculated. (b) Screen of ENZO FDA-approved drug library. One compound showed inhibition above 30% and was taken to further validation. (c) Structure of the hit compound suramin. (d) Dose-response curve with suramin shows an IC₅₀ of 8.7 μM for the SARS-CoV-2 nsp3 macrodomain in the FRET-based. (e) Suramin shows stabilization of SARS-CoV-2 nsp3 macrodomain by DSF. (f) Inhibition profile of suramin against human and viral ADP-ribosyl binders used in this study. The inhibition of was calculated based on the ratiometric FRET signals of the CFP-fused binders mixed with YFP-GAP(MAR) or YFP-GAP(PAR).

### EMBODIMENTS

“ADP-ribosylation” including both “MARylation” and “PARylation”, is catalyzed by an enzyme such as ADP-ribosyltransferases including poly(ADP-ribose)polymerase (PARPs), arginine-specific ecto-enzymes such as ARTC1-6 and a lot of bacterial toxins. Examples of ADP-ribosyltransferases include, in humans, PARPs, and in bacteria, a bacterial toxin DarT.

“MARylated” means when ADP-ribosylation results in the transfer of a single mono(ADP-ribose) (MAR) group on a protein or nucleic acid.

“PARylated” means when ADP-ribosylation results in the transfer of multiple ADP-ribose (ADPr) group on a protein or nucleic acid.

The term “PARP family enzyme” or “PARP” refers to poly (ADP-ribose) polymerases (PARPs) which are a family of related enzymes that share the ability to catalyze the transfer of ADP-ribose to target proteins. PARPs play an important role in various cellular processes, including modulation of chromatin structure, transcription, replication, recombination, and DNA repair.

The term “G alpha subunit” or “Gαi” refers herein to one of the three types of subunits (i.e. alpha (α), beta (β) and gamma (γ) subunits) of G proteins, which are membrane-associated, heterotrimeric G proteins. G proteins, also known as guanine nucleotide-binding proteins, are a family of proteins that act as molecular switches inside cells, and are involved in transmitting signals from a variety of stimuli outside a cell to its interior. An example of human G alpha subunit sequence is shown in SEQ ID NO:4.

The terms “polypeptide”, “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide”, “peptide” and “protein” include glycoproteins, as well as non-glycoproteins.

The term “signal” refers herein to any physical or chemical effect. The signal may be for example a luminous signal, for example a fluorescent, luminescent, colorimetric or electric, this list not being limiting.

The term “luminescent protein” or “luminescent label” refers to an entity or domain which has the property of releasing, in the form of photons with an energy of nonthermal origin, a part of the energy absorbed during an excitation. It therefore involves the deactivation of an excited molecule toward a lower energy state. In other words, a luminescent molecule is a molecule capable of acting on an appropriate substance in order to generate luminescence. Advantageously, the luminescent protein or label has the property of emitting blue, yellow or green light. The luminescent protein can be chosen from among those known to those skilled in the art.

The term “fluorescent label” refers to a molecule having the property of absorbing the light energy (excitation light) and the restore rapidly in the form of fluorescent light, by emission of a photon in a very rapid manner (emission light). Once the energy of the photon absorbed, the molecule is then generally in a state electronically energized. In other words, it may be a fluorophore or a fluorochrome.

The term “FRET” refers to fluorescent resonance energy transfer processes that occur between two chromophores. The chromophores as used herein comprise, for example, fluorescent, luminescent and other non-fluorescent components.

The term “BRET” refers to Resonance Energy Transfer (RET) between a bioluminescent donor moiety (i.e. a BRET energy donor) and a fluorescent acceptor moiety (i.e. a BRET energy acceptor).

The term “tag” as used herein is meant to be understood in its broadest sense and to include, but is not limited to any suitable enzymatic, fluorescent, or radioactive labels and suitable epitopes, including but not limited to biotin tag, HA-tag, Myc-tag, T7, His-tag, FLAG-tag, Calmodulin binding proteins, glutathione-S-transferase, strep-tag, KT3-epitope, EEF-epitopes, green-fluorescent protein and variants thereof. A “tag” can also be any means to bind and/or immobilize a protein, such as the MARylated or PARylated protein of the present invention, to a surface.

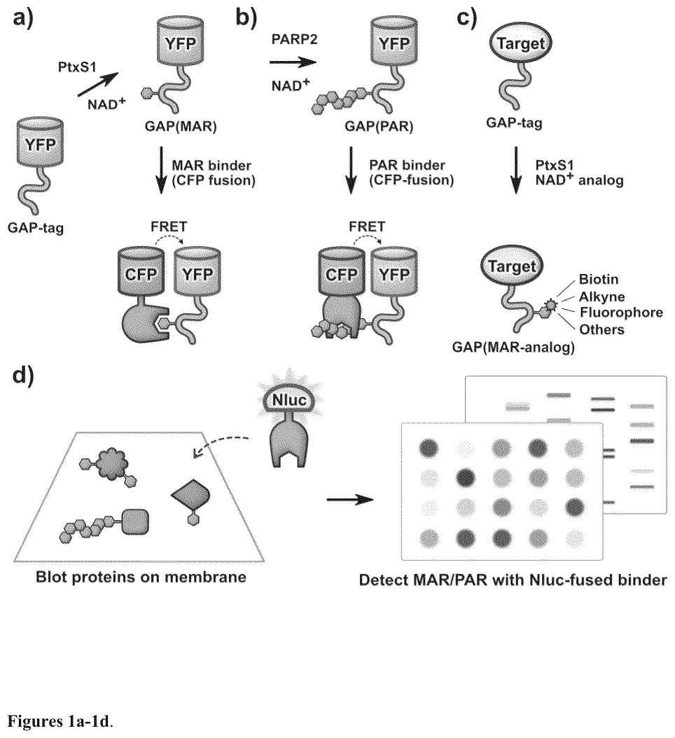

The term “domain” can be interpreted herein to encompass functional amino acid sequences in a polypeptide, such as sequences for binding or target sites for post-translational modifications, which retain their function when incorporated to a fusion protein. The present invention provides a system for in vitro studies that allows for simple and efficient setup of binding assays for ADP-ribosyl readers and erasers based on site-specific cysteine ADP-ribosylation. We extended this system and demonstrated the ability to modify proteins at a C-terminal peptide tag with chemically modified NAD⁺-analogs. This method open ways for the development of various in-vitro assay systems (FIG. 1). To show the applicability for screening, we set up a binding assay for the macrodomain of SARS-CoV-2 non-structural protein 3 and identified the FDA approved drug suramin as moderate inhibitor.

Specifically, the present invention is directed to a method for detecting binding to an ADP-ribosyl group or a polymer thereof, wherein said group or polymer is coupled to a peptide or protein, the method comprising the steps of:
i) providing a first entity comprising a first label or tag, said entity comprising an amino acid sequence comprising a cysteine residue whereto at least one ADP-ribosyl group or an analog thereof is coupled via an S-glycosidic bond;
ii) contacting in an assay said first entity with a second entity, said second entity being or suspected of being capable of binding to an ADP-ribosyl group or polymer thereof coupled to a peptide or protein; and
iii) measuring a signal derived from said first label or localized by said tag, wherein said assay is arranged so that the signal detected is different or is localized differently when said second entity binds to said at least one ADP-ribosyl group of the first entity from the signal detected when the binding interaction between said second entity and said ADP-ribosyl group has not occurred.

In a preferred embodiment, said amino acid sequence corresponds to the C-terminal sequence of a G alpha subunit of G proteins, preferably of SEQ ID NO:4, or has at least 75% sequence identity with the C-terminal sequence of said G alpha subunit and wherein, preferably, said amino acid sequence corresponding to the C-terminal sequence of said G alpha subunit or having at least 75% sequence identity with the C-terminal sequence of heterotrimeric G proteins is at least 4, and more preferably up to 50, 60, 70, 80, 90, 100, 150, 200, 250 or 300 amino acids long sequence or peptide.

In another preferred embodiment, the coupling of said at least one ADP-ribosyl group to said cysteine residue via said S-glycosidic was catalyzed by a pertussis toxin and in case of a polymer preferably extended by a PARP family enzyme.

In further preferred embodiments, said first entity comprises at least 4 amino acid long C-terminal sequence CGLF (SEQ ID NO:1) or CGLY (SEQ ID NO:2) corresponding to the C-terminal sequence of G alpha subunit of G proteins, and wherein at least one ADP-ribosyl group is coupled to the cysteine (C) of SEQ ID NO:1 or SEQ ID NO:2 via a S-glycosidic bond.

In a particular preferred embodiment, said first entity comprises C-terminal sequence KX₁NLKX₂CGLX₃ (SEQ ID NO:3), wherein X₁ is E or N, X₂ is E or D, and X₃ is F or Y.

In further preferred embodiments, said first entity is a fusion protein preferably comprising without limitation a luminescent or fluorescent protein domain or entity.

In further preferred embodiments, said first entity is a fusion protein comprising without limitation a binding or enzymatic tag such as GST-tag or a digoxigenin tag.

In a particular preferred embodiment, said second entity comprises a biomacromolecule capable of binding to said ADP-ribosyl group or a polymer thereof coupled to a peptide or protein.

In further preferred embodiments, said biomacromolecule is selected without limitation from a group consisting of macro domains, ADP-ribosyl-acceptor hydrolase (ARH) family proteins, BRCA1 C-terminal (BRCT) domains, Poly(ADP-ribose)-binding zinc finger motifs, and a conserved globular WWE domain of poly-ADP-ribose polymerase homologs.

In a particular preferred embodiment, said second entity comprises a second label and in step iii) the signal(s) derived from the first and second labels is/are measured, wherein the signal(s) detected is/are different or differently localized when said second entity binds to said at least one ADP-ribosyl group of the first entity from the signal(s) detected when the binding interaction between said second entity and said ADP-ribosyl group has not occurred.

In another preferred embodiment, said first and second labels are distinct luminescent or fluorescent labels.

In a particular preferred embodiment, a candidate inhibitor compound is also added to the assay in step ii), wherein said candidate inhibitor is known or suspected to inhibit the binding interaction between said second entity and said ADP-ribosyl group or polymer thereof coupled to said first entity.

In another embodiment, said candidate inhibitor compound is found to be an inhibitor of the ADP-ribosyl binding if said binding interaction is inhibited in the assay in the presence of said candidate inhibitor but not in the absence of said candidate inhibitor.

In a particular preferred embodiment, the method of the invention comprises the steps of:
i) providing a first luminescent or fluorescent fusion protein comprising an amino acid sequence comprising a cysteine residue whereto at least one ADP-ribosyl group is coupled via an S-glycosidic bond;
ii) contacting in an assay said first luminescent or fluorescent fusion protein with a second luminescent or fluorescent fusion protein capable of binding an ADP-ribosyl group and with a candidate inhibitor compound; and
iii) measuring luminescence or fluorescence signals from said assay, wherein the signal detected is different when the second fusion protein binds to said at least one ADP-ribosyl group of the first fusion protein from the signal detected when the interaction between said second fusion protein and said ADP-ribosyl group is inhibited by the presence of said candidate inhibitor compound, wherein said candidate inhibitor compound is found to be an inhibitor of ADP-ribosyl binding if said binding interaction is inhibited in the assay in the presence of said candidate inhibitor but not in the absence of said candidate inhibitor.

In a particular preferred embodiment, said first and second fusion proteins comprise a fluorescent protein selected from a group consisting of: GFP (“Green Fluorescent Protein”), YFP (“Yellow Fluorescent Protein”), CFP (“Cyan Fluorescent Protein), eYFP (“Enhanced Yellow Fluorescent Protein”, eCFP (“Enhanced Cyan Fluorescent Protein”), derivatives and variants thereof, so that the fluorescent protein of the first fusion protein is preferably distinct from the fluorescent protein of the second fusion protein.

In further preferred embodiments, the method of the invention comprises initial steps of:
providing an entity coupled to said first label or tag, said entity comprising at least 4 amino acid long amino acid sequence CGLF (SEQ ID NO:1) or CGLY (SEQ ID NO:2) corresponding to the C-terminal sequence of the G alpha subunit of G proteins; and
incorporating at least one ADP-ribosyl group derived from NAD⁺ or an analog thereof to the cysteine residue of SEQ ID NO:1 or 2 utilizing a pertussis toxin to obtain said first entity; and
optionally extending said ADP-ribosyl group coupled to said first entity to a polymer by using a PARP enzyme.

The present invention also provides a kit for detecting binding to an ADP-ribosyl group or a polymer thereof, wherein said group or polymer is coupled to a peptide or protein, the kit comprising
in a first container: a first entity comprising a first label or tag, said entity comprising an amino acid sequence comprising a cysteine residue whereto at least one ADP-ribosyl group is coupled via an S-glycosidic bond, and
in a second container: a second entity coupled to a second label or tag, wherein said second entity is capable of binding to an ADP-ribosyl group or a polymer thereof coupled to a peptide or protein.

In a preferred embodiment of the kit, said amino acid sequence preferably corresponds to the C-terminal sequence of a G alpha subunit of G proteins, preferably of SEQ ID NO:4, or has at least 75% sequence identity with the C-terminal sequence of a G alpha subunit of G proteins and wherein, more preferably, said C-terminal sequence corresponding to the C-terminal sequence of a G alpha subunit of G proteins or having at least 75% sequence identity with the C-terminal sequence of a G alpha subunit of G proteins is at least 4, and more preferably up to 50, 60, 70, 80, 90, 100, 150, 200, 250 or 300 amino acids long sequence or peptide, most preferably of the C-terminal of SEQ ID NO:4 or a sequence having at least 75% sequence identity thereto.

In another preferred embodiment, said first and second labels can be without limitation distinct luminescent or fluorescent labels.

In a particular preferred embodiment, said first entity is a fusion protein comprising a binding or enzymatic tag such as GST-tag or a digoxigenin tag.

In a particular preferred embodiment, said first and second entities are fusion proteins comprise without limitation a green fluorescent protein (GFP) or a derivative or variant thereof.

In a particular preferred embodiment, said first entity comprises at least 4 amino acid long amino acid sequence CGLF (SEQ ID NO:1) or CGLY (SEQ ID NO:2) corresponding to the C-terminal sequence of a G alpha subunit of G proteins, and wherein at least one ADP-ribosyl group is coupled to the cysteine (C) of SEQ ID NO:1 or SEQ ID NO:2 via an S-glycosidic bond.

In another preferred embodiment, said first entity comprises C-terminal sequence KX₁NLKX₂CGLX₃ (SEQ ID NO:3), wherein X₁ is E or N, X₂ is E or D, and X₃ is F or Y.

In a particular preferred embodiment, said second entity comprises a biomacromolecule capable of binding to said ADP-ribosyl group or polymer thereof coupled to a peptide or protein.

In a particular preferred embodiment, said biomacromolecule is selected without limitation from a group consisting of macro domains, ARH family proteins, BRCT domains, PAR binding zinc motifs, and WWE domains.

The present invention is also providing a fusion protein comprising a first domain and a second domain, wherein said second domain comprises an amino acid sequence corresponding to the C-terminal sequence of a G alpha subunit of G proteins, preferably of SEQ ID NO:4, or having at least 75% sequence identity with the C-terminal sequence of a G alpha subunit of G proteins, and wherein said amino acid sequence comprises a cysteine residue whereto at least one ADP-ribosyl group or an analog thereof is coupled via an S-glycosidic bond.

In a preferred embodiment of said fusion protein, said C-terminal sequence corresponds to the C-terminal sequence of a G alpha subunit of G proteins, preferably of SEQ ID NO:4 or has at least 75% sequence identity with the C-terminal sequence of a G alpha subunit of G proteins is at least 4 amino acids long sequence or peptide.

In another preferred embodiment, said amino acid sequence of said second domain comprises amino acid sequence CGLF (SEQ ID NO:1) or CGLY (SEQ ID NO:2) corresponding to the C-terminal sequence of a G alpha subunit of G proteins, and wherein at least one ADP-ribosyl group is coupled to the cysteine (C) of SEQ ID NO:1 or SEQ ID NO:2 via an S-glycosidic bond.

In another preferred embodiment, said amino acid sequence of the second domain comprises C-terminal sequence KX₁NLKX₂CGLX₃ (SEQ ID NO:3), wherein X₁ is E or N, X₂ is E or D, and X₃ is F or Y.

In another preferred embodiment of said fusion protein, said first domain is a fluorescent protein preferably selected from a group consisting of: GFP (“Green Fluorescent Protein”), YFP (“Yellow Fluorescent Protein”), CFP (“Cyan Fluorescent Protein), eYFP (“Enhanced Yellow Fluorescent Protein”, eCFP (“Enhanced Cyan Fluorescent Protein”), derivatives and variants thereof.

The present invention is further directed to a system comprising
i) a fusion protein comprising a first domain and a second domain, wherein said second domain comprises an amino acid sequence comprising a cysteine residue whereto at least one ADP-ribosyl group or an analog thereof is coupled via an S-glycosidic bond; and
ii) a biomacromolecule preferably selected from a group consisting of macro domains, ARH family proteins, BRCT domains, PAR binding zinc motifs, and WWE domains, wherein said biomacromolecule specifically recognizes said at least one ADP-ribosyl group or an analog thereof of said fusion protein;
wherein, in said system, said biomacromolecule is bound to said at least one ADP-ribosyl group or an analog thereof of said fusion protein so that said biomolecule and said fusion protein are linked together and form a coupled entity.

In a preferred embodiment of said system, said second domain comprises an amino acid sequence corresponding to the C-terminal sequence of a G alpha subunit of G proteins, preferably of SEQ ID NO:4, or having at least 75% sequence identity with the C-terminal sequence of a G alpha subunit of G proteins.

In a preferred embodiment, said system comprises means to detect the presence of said coupled entity in said system. Said means can preferably be selected from a group of luminescent and fluorescent labels and devices capable of receiving a signal from these labels.

While the following examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated.

Furthermore, it is to be understood that the use of “a” or “an”, that is, a singular form, throughout this document does not exclude a plurality.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

### EXPERIMENTAL SECTION

### Material and Methods

### Cloning

Expression constructs for CFP- or Nanoluciferase-fused proteins were cloned into pNIC28-CFP or pNH-Nluc by sequence and ligation independent cloning. We prepared pNIC28-CFP and pNH-Nluc by insertion of the sequence for CFP or Nanoluciferase between His6-tag and TEV protease cleavage site of pNIC28-Bsa4 or pNH-Trxt. Other protein constructs were cloned into pNIC28-Bsa or pNIC-MBP vectors.

### Protein Expression

The plasmids were transformed to E. coli BL21(DE3) or E. coli Rosetta 2 cells. Terrific Broth (TB) autoinduction media including trace elements (Formedium, Hunstanton, Norfolk, England) was supplemented with 8 g/l glycerol and antibiotics and inoculated with 1:100 of preculture grown over night in LB. The flasks were incubated shaking at 37° C. until an OD600 of about 1 was reached. The temperature was set to 18° C. and incubation continued overnight. The cells were collected by centrifugation at 4,200×g for 30 min at 4° C. The pellets were resuspended in lysis buffer (50 mM HEPES pH 7.5, 500 mM NaCl, 15 mM imidazole). Resuspended cells were stored at −20° C. until purification.

### Protein Purification

All constructs were initially purified by immobilized metal affinity chromatography (IMAC). The cells were thawed and lysed by sonication. The lysate was centrifuged, and the supernatant filtered and loaded onto a 5 ml HiTrap HP column equilibrated with lysis buffer and charged with Ni²⁺. The column was washed with 2-10 column volumes of lysis buffer and 2-10 column volumes of lysis buffer containing 10-50 mM imidazole. Elution was done with elution buffer containing 100-500 mM imidazole. Following IMAC, an additional size exclusion purification was performed for most of the proteins. Finally, proteins were concentrated to a concentration of 0.04-3 mM and subsequently aliquoted and flash frozen in liquid nitrogen and stored at −70° C.

### Preparation of Mono- and Poly-ADP-Ribosylated Gαi Proteins

YFP with C-terminal Gαi-peptide tag was purified by IMAC and dialyzed against 20 mM HEPES pH 7.5, 350 mM NaCl. YFP-Gαi was diluted to 100 μM in 50 mM sodium phosphate buffer pH 7.0 and mixed with 1.5 μM catalytic S1 domain of pertussis toxin and 150 μM sodium β-Nicotinamide adenine dinucleotide. The reaction was incubated for an hour at room temperature. To ensure completeness of the reaction, a second 150 μM were added to the reaction. Incubation was continued for 1 h at room temperature. The reaction mixture loaded to an IMAC column to remove pertussis toxin, hydrolysis products and unreacted NAD⁺. IMAC was carried out as described in the purification procedures above. The buffer was exchanged to 20 mM HEPES pH 7.5, 150 mM NaCl, 0.5 mM TCEP and the MARylated YFP-Gαi was subsequently concentrated to about 1 mM concentration using a Amicon Ultra-15 Centrifugal Filter Unit (MWCO: 10 kDa). The protein was flash frozen in liquid nitrogen and stored at −70° C.

PARylated YFP-Gαi for FRET experiments was prepared from MARylated YFP-Gαi. 10 μM of MARylated YFP-Gαi was incubated in the presence of 400 nM PARP2 (residues 90-583) and 1 mM NAD⁺ in a buffer solution containing 50 mM Tris [pH 8.0] and 5 mM MgCl2 for 2 h at room temperature. The reacted sample was then purified using IMAC as described above to remove PARP2. The sample buffer was exchanged to 30 mM HEPES [pH 7.5], 150 mM NaCl, 10% glycerol, 0.5 mM TCEP using an Amicon Ultra-15 Centrifugal Filter Unit (MWCO: 10 kDa). The protein was aliquoted and flash frozen in liquid nitrogen and stored at −70° C.

For Tankyrase1 PARylated YFP-Gαi production, MARylated YFP-Gαi was incubated with 200 nM Tankyrase 1 SAM-catalytic domain dimer, 1 and 10 mM NAD⁺ in a buffer solution containing 10 mM BisTrisPropane [pH 7.0], 0.01% Triton X-100. The reaction has carried out for 16 h at room temperature.

### Blot-Based Detection of Mono- and Poly-ADP-Ribosylation

For dot blot experiments, we transferred 0.5 μl per spot of the sample solution to dry nitrocellulose membranes using Echo 650. All following steps were performed at room temperature. After drying of the spots, the membrane was blocked on a shaker for 10 min in 15 ml 5% (w/v) skimmed milk powder in TBS-T. The blocking solution was discarded, and the membrane was incubated on a shaker with 15 ml of 0.1 μg/ml Nanoluc-eAf1521 in 1% (w/v) skimmed milk powder in TBS-T. After discarding the Nanoluc-eAf1521 solution, the membrane was rinsed with 15 ml TBS-T and incubated on a shaker with 15 ml TBS-T for 15 min. After a final rinsing with 15 ml TBS-T, the membrane was imaged using 500 μl of 1:1000 NanoGlo substrate (Promega) diluted in 10 mM sodium phosphate buffer pH 7.0.

For western blot, 10 μl samples were first run in SDS-PAGE (Mini-Protean TGX 4-20% gradient gel, BioRad). The proteins were then transferred to a nitrocellulose membrane (Trans-Blot Turbo, BioRad) using TransBlot semi dry system (BioRad). After transfer, membranes were treated following the same procedure as described above for dot-blot. Nanoluc-eAF1521 and Nanoluc-ALC1 were used at 0.1 μg/ml.

### Testing Nluc-eAf1521 and Nluc-ALC1 Sensitivity and Selectivity.

To generate auto-PARylated TNKS1, 10 μM TNKS1 construct was mixed with 1 mM NAD⁺ in 50 mM Bis-Tris-Propane pH 7.0, 0.01% Triton X-100, 0.5 mM TCEP. To generate a control containing TNKS1 without PAR, partial auto-PARylation that occurred during recombinant expression in E. coli was removed by mixing TNKS1 construct with 2 μM snake venom phosphodiesterase I.

Modification Test of YFP-Gαi with 6-Biotin-17-NAD⁺

10 μM YFP-Gαi or YFP-Gαi(mutant) were mixed with 1 μM NAD⁺ or 6-Biotin-17-NAD⁺ (Biolog). Reactions were prepared in absence or presence of 0.5 μM PtxS1 and were incubated for 1 h at room temperature and then blotted to a dry nitrocellulose membrane (0.5 μl per spot of the sample solution to dry nitrocellulose membranes using Echo 650). The membrane was let dry and thereafter blocked on a shaker for 10 min in 15 ml blocking buffer (1% casein in TBS, BioRad). The blocking solution was discarded, and the membrane was incubated on a shaker for 1 hour with 15 ml of 1:5000 Streptavidin-HRP in blocking buffer. After discarding the Streptavidin-HRP solution, the membrane was rinsed with 15 ml TBS-T and incubated on a shaker with 15 ml TBS-T for 15 min. After a final rinsing with 15 ml TBS-T, the membrane was imaged using ECL solution (BioRad).

Modification Test of YFP-Gαi with 6-Parg-NAD⁺ and Addition Cy3 and Cy5 Azides by CuAAC

YFP-Gαi(6-Parg-MAR) was prepared as described above for YFP-Gαi using 6-Parg-NAD⁺ instead of NAD⁺. To test the addition of Cy3/Cy5 to YFP-Gαi(6-PARG-MAR) by CuAAC, reactions were prepared in 25 mM HEPES pH 7.5 by mixing 15 μM of YFP-Gαi(6-PARG-MAR) or YFP-Gαi(MAR) with 10 mM sodium ascorbate, 50 μM Cy3-azide or Cy5-azide and pre-mixed 300 μM CuSO4 and 600 μM L-Histidine. Additionally, controls without protein were prepared. The reactions were let incubate for 3 hours at room temperature and afterwards blotted on a nitrocellulose membrane (5 μl per spot). The membrane was washed in 15 ml TBS-T for 30 min and imaged. Fluorescence imaging was done with an Azure 600 imaging system (Azure Biosystems) using Cy3 or Cy5 filter settings, respectively.

### FRET Measurement

The samples were excited at 410 nm and emission at 477 nm and 527 nm wavelengths were measured. The ratiometric FRET value (rFRET) was calculated by dividing the fluorescence intensity at 527 nm by the fluorescence intensity at 477 nm. The experiments were carried out in assay buffer (10 mM Bis-Tris-Propane pH 7.0, 3% (w/v) PEG20,000, 0.01% (v/v) Triton X-100 and 0.5 mM TCEP) in 10 μl volume per well unless stated otherwise.

### BRET Measurement

The reactions were performed in 384-well white OptiPlates (PerkinElmer). A reaction volume of 40 μl per well was used. 50 nM Nluc-MDO2 were mixed with 1 μM MARylated YFP-Gαi. The reaction was started by addition of 1:4000 NanoGlo substrate (Promega, catalogue number N1110). The reaction was incubated for 5 minutes and the emission was measured at wavelengths of 445-470 nm and 520-545 nm using Tecan Spak multimode plate reader with luminescence readout and a settle time of 10 ms and integration time of 100 ms. The ratiometric BRET value (rBRET) was calculated by dividing the luminescence intensity and 520-545 nm by the luminescence intensity at 445-470 nm. The experiments were carried out in assay buffer (10 mM Bis-Tris-Propane pH 7.0, 3% (w/v) PEG20,000, 0.01% (v/v) Triton X-100 and 0.5 mM TCEP).

### AlphaScreen

The reaction was performed in a 384 well flat-grey Alphaplate (PerkinElmer) in a total volume of 25 μl. The reaction consisted of 300 nM His-tagged Gαi(MAR) mixed with 300 nM Bio-MDO2 in a buffer containing (25 mM HEPES pH 7.5, 100 mM NaCl, and 0.1 mg/ml BSA). The plate was sealed and incubated for 80 min at RT with constant shaking at 300 rpm. Finally, 5 μg/ml nickel chelate acceptor and streptavidin donor beads were added to the plates followed by additional 3 hrs incubation. The plate contained blank wells (assay buffer and AlphaScreen beads only), control 1 (Bio-MDO2, His-Gαi) and control 2 (Bio-MDO2, modified His-Gαi and ADPr). Luminescence was read using Tecan infinite M1000 Pro plate reader with AlphaScreen detection module.

### Biolayer Interferometry

Biolayer interferometry (BLI) assays were carried out in Octet Red system (Forte Bio) in a buffer containing 10 mM BisTrisPropane [pH7.0], 150 mM NaCl, 1% BSA, 0.02% TritonX-100 and at 30° C. and shaking at 1500 rpm. 10 μg/ml YFP-Gαi or MARylated YFP-Gαi was loaded on Ni²⁺-NTA coated sensors, followed by a wash step in buffer. Association to MDO2 was measured by dipping the sensors in solution containing 0-2 μM MDO2 for 120 s, while for the dissociation step the sensors were dipped in buffer for 120 s.

For the ADP-ribose competition experiments, 10 μg/ml YFP-Gαᵢ or MARylated YFP-Gαᵢ were loaded onto Ni2+-NTA coated sensors. For association, sensors were dipped in 100 nM MDO2 mixed with a half-log dilution series of ADPr (10 nM to 10 μM) for 120 s and then transferred to buffer for the dissociation step.

### Cysteine-ADP-Ribosyl Hydrolysis Assay

All samples were incubated for 24 hours at room temperature prior to blotting. 1 μM of CFP-fused proteins or 0.01, 0.1 and 1 μM of SVP were mixed with 10 μM MARylated YFP-Gαi in 10 mM HEPES pH 7.5, 25 mM NaCl, 0.5 mM TCEP. After incubation, 0.5 μl per spot of the reaction mixtures were blotted next to 0.5 μl spots of 10 μM MARylated YFP-Gαi. As control, 10 μM of non-MARylated YFP-Gαi was blotted next to 10 μM of MARylated YFP-Gαi.

### Validatory Screening of FDA-Approved Drug Library

For the screening, 40 nl of 10 mM compound stocks dissolved in DMSO from the FDA-approved drug library (Enzo Life Sciences) were transferred to 384-well black low-volume polypropylene plates (Fisherbrand). The sample mixture containing 1 μM CFP-SARS-CoV-2 nsp3 macrodomain and 5 μM MARylated YFP-Gαi was prepared in assay buffer (10 mM Bis-Tris-Propane pH 7.0, 3% (w/v) PEG20,000, 0.01% (v/v) Triton X-100 and 0.5 mM TCEP) and 20 μl per well were dispensed using Mantis (Formulatrix). The rFRET signal was determined after 5 minute incubation time. The sample mixtures in presence or absence of 200 μM ADP-ribose were used as positive and negative controls, respectively.

### Validation of Suramin Binding by DSF

The SARS-CoV-2 nsp3 macrodomain without tags was diluted to 5 μM in 10 mM HEPES pH 7.5, 25 mM NaCl, 0.5 mM TCEP buffer and mixed with 5×SYPRO Orange. Samples were prepared with 10 μM, 50 μM, 100 μM or 1 mM of suramin. Samples in presence or absence of 1 mM ADP-ribose were used as controls. Samples were transferred to 96-well qPCR plates. Measurement was performed in a BioRad C1000 CFX96 thermal cycler. Data points for melting curves were recorded in 1 min intervals from 20-95° C., with the temperature increasing by 1° C./min. The analysis of the data was done in GraphPad Prism 7 using a nonlinear regression analysis (Boltzmann sigmoid equation) of normalized data.

### Example 1. Initial Preparation of Proteins for Toolbox Studies

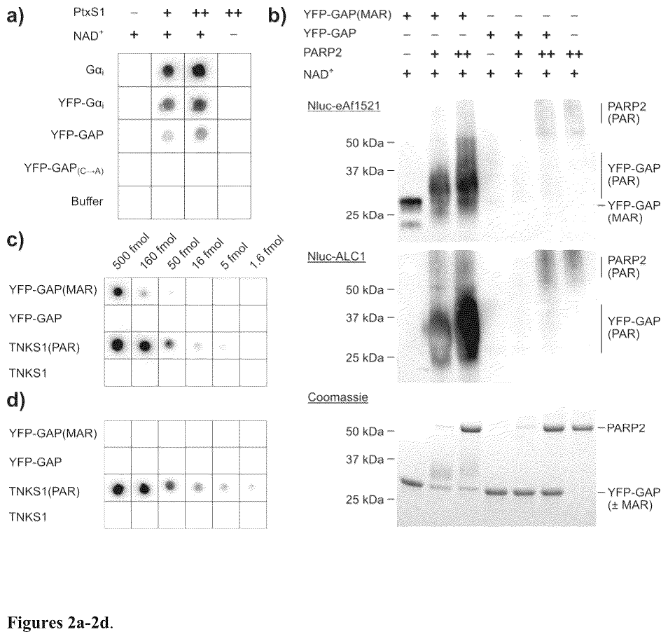

All proteins used in this study were recombinantly produced in E coli. As a tool to detect ADP-ribosylation in blot-based methods, we produced the recently reported ADP-ribosyl superbinder eAf1521 (Nowak et al., 2020) as fusion protein with nanoluciferase. We found that this protein works in a simple and fast one-step protocol for sensitive detection of mono- and poly-ADP-ribosylated proteins (FIGS. 2c and 2d).

We reasoned that mono-ADP-ribosylation of Gαi by pertussis toxin would provide a good probe to test binding to ADP-ribosyl because the modification is well-defined at a single residue and it uses cysteine to produce a stable S-glycosidic linkage. To produce a mono-ADP-ribosylated Gαi probe, we tested the ability of pertussis toxin to modify only the 10-amino acid C-terminal peptide of Gαi when fused to YFP compared to full-length Gαi variants (FIG. 2a). We found that the fused peptide could be modified effectively.

Next, we aimed to introduce a poly-ADP-ribosyl chain to the Gαi peptide. We reasoned that the mono-ADP-ribosyl group of the previously modified Gαi peptide could serve as starting point for elongation to poly-ADP-ribose by PARP enzymes. After optimization of the reaction conditions, we found that both PARP2 was able to generate poly-ADP-ribosylated Gαi constructs (FIG. 2b).

### Example 2. The Gαi Peptide as Tag for Site Specific Labelling with NAD⁺-Analogs

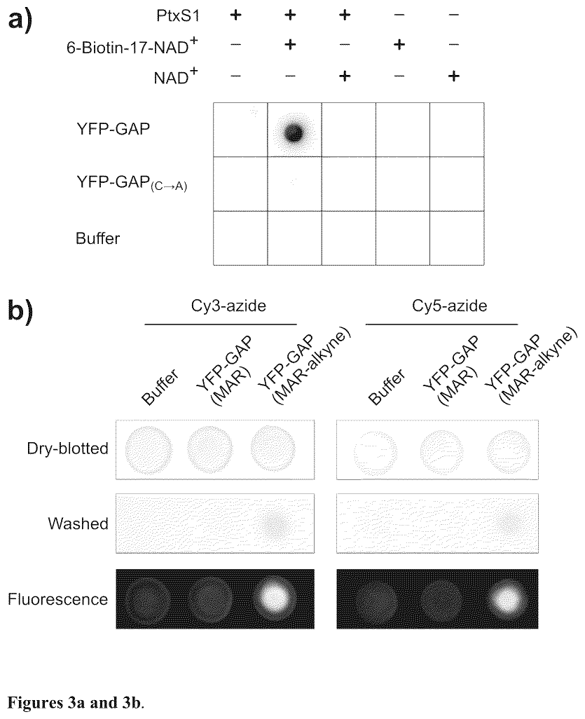

While we could use the Gαi peptide tag to site-specifically label proteins with accessible C-terminus with ADP-ribose, we reasoned that this system could be extended to introduce chemical NAD⁺ analogs to the C-terminus of proteins. Many NAD⁺ analogs already exist and are commercially available such as biotinylated, fluorescent and click-chemistry ready NAD⁺ analogs. We first tested the modification of the YFP-Gαi-peptide tagged protein with a biotinylated NAD⁺ analog. The site specific modification was detected using dot blot with streptavidin-conjugated horseradish peroxidase (FIG. 3a).

We further tested modification of the YFP-fused Gαi peptide tag with 6-propargyladenine-NAD⁺ which allows for copper(I)-catalyzed alkyne-azide cycloaddition of azide-groups (FIG. 3b). We show by dot blot that the azide-group containing fluorescent dyes Cy3 and Cy5 could be added to the protein containing the Gαi peptide modified with 6-propargyladenine-NAD⁺, while Cy3 and Cy5 were not added to the Gαi peptide modified with canonical NAD⁺.

### Example 3. Evaluation of Binding with Confirmed and Potential ADP-Ribosyl Binders

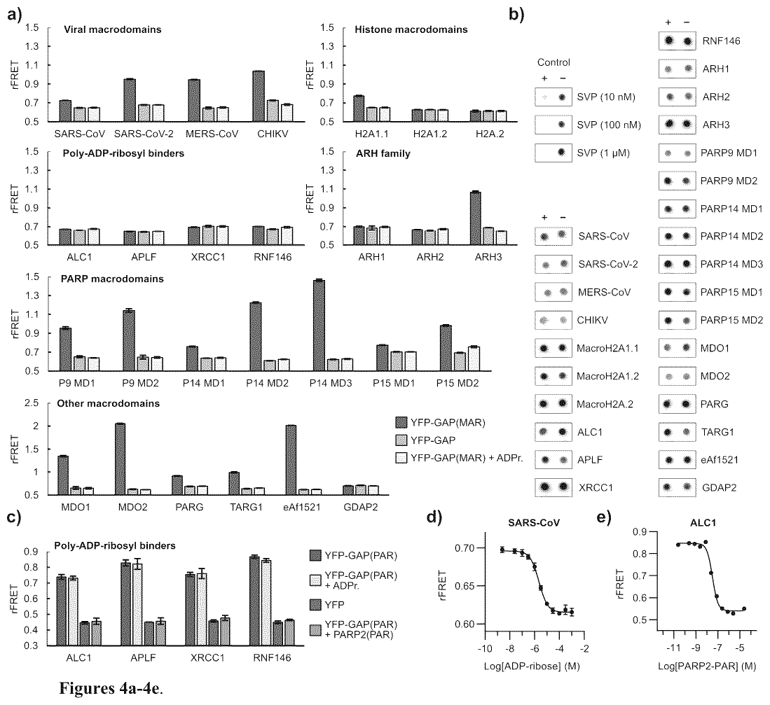

We selected a set of 27 different proteins to be tested for binding to the MARylated Gαi. We recombinantly produced these proteins in E. coli as fusions with CFP and tested ratiometric FRET signals upon binding to the MARylated YFP-Gαi construct (FIG. 4a). We used non-MARylated YFP-Gαi construct as a control as well as MARylated YFP-Gαi containing 200 μM ADP-ribose to compete with the interaction. A higher FRET signal correlates with a higher occupancy and thus binding-affinity of the binding partners but is also affected by the distance and orientation of the fluorophores. We found that the proteins that were previously reported to bind ADP-ribose showed a higher FRET signal compared to the controls, indicating the binding to the MARylated probe. ARH1 is known to bind ADP-ribose with a low affinity, which is likely the reason that we could not measure a FRET signal for this construct. While all three histone macrodomains macroH2A1.1, macroH2A1.2 and macroH2A2 have highly similar sequence identities, only macroH2A1.1 has the ability to bind ADP-ribose. This is in agreement with our observations, wherein only macroH2A1.1 shows an elevated FRET signal over the controls.

One could expect that binding to cysteine-ADP-ribosyl groups would be not physiologically relevant for some or most of the ADP-ribosyl binding proteins tested. It is surprising that none of the binders seem to display high specificity towards either O- or N-glycosidically linked ADP-ribose and that the cysteine sulphur did not abolish binding in any of the confirmed ADP-ribosyl binders tested.

While cysteine-ADP-glycosylhydrolase activity was detected in human erythrocytes and mitochondria, no specific human enzymes have been identified to date that have the ability to hydrolyse the S-glycosidic bond of cysteine-ADP-ribose. We mixed the CFP-fused constructs from above with mono-ADP-ribosylated YFP-Gαi and tested hydrolysis of ADP-ribose using dot blot after a 24 hour incubation period (FIG. 4b). While the control including snake venom phosphodiesterase I showed loss of the signal through cleavage of the ADP-ribose diphosphate bond, none of the tested proteins were able to hydrolyse the S-glycosidic bond under the conditions tested. These findings highlight the versatility of this system for measuring the ADP-ribosyl binding of proteins that can hydrolyse O- or N-glycosidic linkages such as MDO1, TARG1, ARH3 or SARS-CoV-2 nsp3. We next tested the ratiometric FRET signals of CFP-fused poly-ADP-ribosyl binders when mixed with YFP-Gαi containing a poly-ADP-ribosyl group, which was prepared as described in (FIG. 2b). The poly-ADP-ribosyl binders show an increased ratiometric FRET signal with PARylated YFP-Gαi over the controls, suggesting binding to PAR, but not MAR groups (FIG. 4c). Dose-response curves of representative mono- or poly-ADP-binders show the good signal quality (FIG. 4d, FIG. 4e).

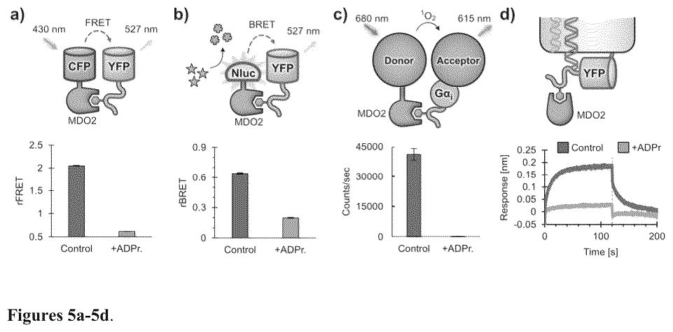

We used FRET for testing the ADP-ribosyl binding above, however we showed that binding to MARylated Gαi could also be measured with different binding technologies. We utilized MDO2 as example protein for this. Similar to the binding of CFP-fused MDO2 to MARylated YFP-Gαi to measure FRET (FIG. 5a), we used Nanoluc-fused MDO2 to generate a BRET signal upon interaction with MARylated YFP-Gαi (FIG. 5b).

While AlphaScreen protocols for ADP-ribosyl readers or erasers exist, we could use our system adapted to AlphaScreen technology to directly probe binding to MARylated Gαi potentially by any reader or eraser as shown for the example of the otherwise hydrolysing MDO2 domain (Figure Sc).

We further showed binding using biolayer interferometry (BLI, FIG. 5d), which in recent years has gained popularity as method for screening of small molecule compounds. This method also allows for simple quantification of kinetic binding parameters such as the dissociation constant.

### Example 4. Screening for Inhibitors Against SARS-CoV-2 Nsp3 Macrodomain

In light of the current situation regarding the COVID-19 pandemic, we chose to demonstrate the applicability of this binding system for screening of small molecule inhibitors against the macrodomain of SARS-CoV-2 nsp3. Currently, efforts are being made by researchers worldwide to find inhibitors against this macrodomain. The nsp3 macrodomain of coronaviruses was shown to be critical for the viral replication, and small molecule inhibitors might show promise as therapeutic agents to fight infections caused by SARS-CoV-2 (COVID-19) and other viruses.

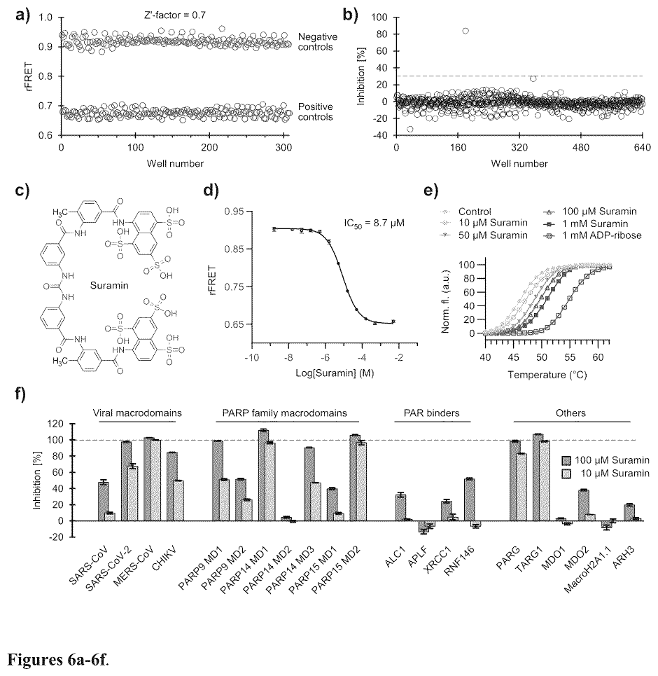

We assessed the quality of the FRET signals of the alternating positive and negative controls in a 384-well plate by mixing CFP-fused SARS-CoV-2 nsp3 macrodomain with MARylated YFP-Gαi in the absence and presence of 200 μM ADP-ribose (FIG. 6a). While the SARS-CoV-2 macrodomain was reported to have a relatively low binding affinity to ADP-ribose (Kd=17 μM), we still found that the signal showed sufficient separation of positive and negative controls. The Z′-factor was calculated to be 0.7, indicating that this assay is suitable for high-throughput screening.

We screened against the ENZO FDA-approved drug library comprising 640 small molecule compounds at 20 μM compound concentration (FIG. 6b). From the screening, only the compound suramin was regarded as hit with 82% inhibition and showed an IC₅₀ of 8.7 μM against the SARS-CoV-2 nsp3 macrodomain when tested with the FRET-based assay (FIG. 6c, FIG. 6d). To confirm binding of the compound to the SARS-CoV-2 nsp3 macrodomain, we performed DSF analysis and showed stabilization of the protein in a concentration dependent manner (FIG. 6e). Intriguingly, suramin is used as a broadband antiviral and antiparasitic drug and was reported to inhibit SARS-CoV2 infection in cell-culture based models. After reviewing the literature associated with suramin, we found that is reported to inhibit a plethora of target proteins ranging from DNA- and RNA-polymerases to sirtuins, ATPases and G protein-coupled receptors, indicating that it exhibits low target specificity. We next tested suramin for inhibition of the viral and human ADP-ribose readers and erasers produces in this study (FIG. 6f). Unsurprisingly, suramin showed strong inhibition even at 10 μM against many of the proteins tested, confirming the low target specificity of this compound.

### REFERENCES

Nowak K, Rosenthal F, Karlberg T, Butepage M, Thorsell A-G, Dreier B, Grossmann J, Sobek J, Imhof R, Luscher B, Schuler H, Pluckthun A, Leslie Pedrioli D M, Hottiger M O. (2020) Engineering Afl521 improves ADP-ribose binding and identification of ADP-ribosylated proteins. Nat. Commun. 11:5199, https://doi.org/10.1038/s41467-020-18981-w
Teloni F and Altmeyer M. (2016) Readers of poly(ADP-ribose): designed to be fit for purpose. Nucleic Acids Res. 44:993-1006, doi: 10.1093/nar/gkv1383
Wazir S, Maksimainen M M, Alanen H I, Galera-Prat A, Lehtiö L. (2021) Activity-Based Screening Assay for Mono-ADP-Ribosylhydrolases. SLAS Discovery 26:67-76, doi: 10.1177/2472555220928911

1. A method for detecting binding to an ADP-ribosyl group or a polymer thereof, wherein said group or polymer is coupled to a peptide or protein, the method comprising the steps of:
i) providing a first entity comprising a first label or tag, said entity comprising an amino acid sequence comprising a cysteine residue, wherein at least one ADP-ribosyl group or an analog thereof is coupled via an S-glycosidic bond;
ii) contacting in an assay said first entity with a second entity, said second entity being or suspected of being capable of binding to an ADP-ribosyl group or polymer thereof coupled to a peptide or protein; and
iii) measuring a signal derived from said first label or localized by said tag, wherein the signal detected is different or is localized differently when said second entity binds to said at least one ADP-ribosyl group of the first entity from the signal detected when the binding interaction between said second entity and said ADP-ribosyl group has not occurred.

2. The method according to claim 1, wherein said amino acid sequence corresponds to the C-terminal sequence of a G alpha subunit of G proteins or has at least 75% sequence identity with the C-terminal sequence of said G alpha subunit and wherein said amino acid sequence corresponding to the C-terminal sequence of said G alpha subunit or having at least 75% sequence identity with the C-terminal sequence of heterotrimeric G proteins is at least a 4 amino acid-long sequence or peptide.

3. The method according to claim 1, wherein the coupling of said at least one ADP-ribosyl group to said cysteine residue via said S-glycosidic bond was catalyzed by a pertussis toxin and in case of said polymer, said ADP-ribosyl group coupled to said cysteine residue was extended by a PARP family enzyme.

4. The method according to claim 1, wherein said first entity comprises at least a 4 amino acid-long C-terminal sequence CGLF (SEQ ID NO:1) or CGLY (SEQ ID NO:2) corresponding to the C-terminal sequence of G alpha subunit of G proteins, and wherein at least one ADP-ribosyl group is coupled to the cysteine (C) of SEQ ID NO:1 or SEQ ID NO:2 via a S-glycosidic bond.

5. The method according to claim 4, wherein said first entity comprises C-terminal sequence KX₁NLKX₂CGLX₃ (SEQ ID NO:3), wherein X₁ is E or N, X₂ is E or D, and X₃ is F or Y.

6. The method according to claim 1, wherein said first entity is a fusion protein comprising a luminescent or fluorescent protein domain or entity.

7. The method according to claim 1, wherein said first entity is a fusion protein comprising a binding or enzymatic tag.

8. The method according to claim 1, wherein said second entity comprises a biomacromolecule capable of binding to said ADP-ribosyl group or a polymer thereof coupled to a peptide or protein.

9. The method according to claim 8, wherein said biomacromolecule is selected from the group consisting of macro domains, ARH family proteins, BRCT domains, PAR binding zinc motifs, and WWE domains.

10. The method according to claim 1, wherein said second entity comprises a second label and in step iii) the signal(s) derived from the first and second labels is/are measured, and wherein the signal(s) detected is/are different or differently localized when said second entity binds to said at least one ADP-ribosyl group of the first entity from the signal(s) detected when the binding interaction between said second entity and said ADP-ribosyl group has not occurred.

11. The method according to claim 10, wherein said first and second labels are distinct luminescent or fluorescent labels.

12. The method according to claim 1, wherein in step ii) a candidate inhibitor compound is also added to the assay, and wherein said candidate inhibitor is known or suspected to inhibit the binding interaction between said second entity and said ADP-ribosyl group or polymer thereof coupled to said first entity.

13. The method according to claim 12, wherein said candidate inhibitor compound is found to be an inhibitor of the ADP-ribosyl binding if said binding interaction is inhibited in the assay in the presence of said candidate inhibitor but not in the absence of said candidate inhibitor.

14. The method according to claim 13, comprising the steps of:
i) providing a first luminescent or fluorescent fusion protein comprising an amino acid sequence comprising a cysteine residue, wherein at least one ADP-ribosyl group is coupled via an S-glycosidic bond;
ii) contacting in an assay said first luminescent or fluorescent fusion protein with a second luminescent or fluorescent fusion protein capable of binding an ADP-ribosyl group and with a candidate inhibitor compound; and
iii) measuring luminescence or fluorescence signals from said assay, wherein the signal detected is different when the second fusion protein binds to said at least one ADP-ribosyl group of the first fusion protein from the signal detected when the interaction between said second fusion protein and said ADP-ribosyl group is inhibited by the presence of said candidate inhibitor compound, wherein said candidate inhibitor compound is found to be an inhibitor of ADP-ribosyl binding if said binding interaction is inhibited in the assay in the presence of said candidate inhibitor but not in the absence of said candidate inhibitor.

15. The method according to claim 14, wherein said first and second fusion proteins comprise a green fluorescent protein (GFP) or a derivative thereof.

16. The method according to claim 1, comprising initial steps of:
providing an entity coupled to said first label or tag, said entity comprising at least a 4 amino acid long amino acid sequence CGLF (SEQ ID NO:1) or CGLY (SEQ ID NO:2) corresponding to the C-terminal sequence of the G alpha subunit of G proteins; and
incorporating at least one ADP-ribosyl group derived from NAD⁺ or an analog thereof to the cysteine residue of SEQ ID NO:1 or 2 utilizing a pertussis toxin to obtain said first entity; and
optionally extending said ADP-ribosyl group coupled to said first entity to a polymer by using a PARP enzyme.

17. A kit for detecting binding to an ADP-ribosyl group or a polymer thereof, wherein said group or polymer is coupled to a peptide or protein, the kit comprising:
in a first container: a first entity comprising a first label or tag, said entity comprising an amino acid sequence comprising a cysteine residue, wherein at least one ADP-ribosyl group is coupled via an S-glycosidic bond, and
in a second container: a second entity coupled to a second label or tag, wherein said second entity is capable of binding to an ADP-ribosyl group or a polymer thereof coupled to a peptide or protein.

18-21. (canceled)

22. The kit according to claim 17, wherein said first entity comprises at least a 4 amino acid long amino acid sequence CGLF (SEQ ID NO:1) or CGLY (SEQ ID NO:2) corresponding to the C-terminal sequence of a G alpha subunit of G proteins, and wherein at least one ADP-ribosyl group is coupled to the cysteine (C) of SEQ ID NO:1 or SEQ ID NO:2 via an S-glycosidic bond.

23. The kit according to claim 22, wherein said first entity comprises C-terminal sequence KX₁NLKX₂CGLX₃ (SEQ ID NO:3), and wherein X₁ is E or N, X₂ is E or D, and X₃ is F or Y.

24-25. (canceled)

26. A fusion protein comprising a first domain and a second domain, wherein said second domain comprises an amino acid sequence corresponding to the C-terminal sequence of a G alpha subunit of G proteins or having at least 75% sequence identity with the C-terminal sequence of a G alpha subunit of G proteins, and wherein said amino acid sequence comprises a cysteine residue, wherein at least one ADP-ribosyl group or an analog thereof is coupled via an S-glycosidic bond.

27-33. (canceled)

34. A system comprising:
i) the fusion protein according to claim 26; and
ii) a biomacromolecule selected from the group consisting of macro domains, ARH family proteins, BRCT domains, PAR binding zinc motifs, and WWE domains, wherein said biomacromolecule specifically recognizes said at least one ADP-ribosyl group or an analog thereof of said fusion protein;
wherein, in said system, said biomacromolecule is bound to said at least one ADP-ribosyl group or an analog thereof of said fusion protein so that said biomolecule and said fusion protein are linked together and form a coupled entity.

35-39. (canceled)


SEQUENCE LISTING (4 sequences)

SEQ ID NO: 1 - protein, 4 aa, Homo sapiens
CGLF

SEQ ID NO: 2 - protein, 4 aa, Homo sapiens
CGLY

SEQ ID NO: 3 - protein, 10 aa, Homo sapiens
  VARIANT 2: Xaa is Glu or Asn
  VARIANT 6: Xaa is Glu or Asp
  VARIANT 10: Xaa is Phe or Tyr
KXNLKXCGLX

SEQ ID NO: 4 - protein, 354 aa, Homo sapiens
MGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF